(19) United States
(12) Patent Application Publication
(10) Pub. No.: US 2025/0037851 A1
(43) Pub. Date: Jan. 30, 2025

(54) METHOD, SYSTEM, AND USER INTERFACE FOR ARTIFICIAL INTELLIGENCE BASED TROUBLESHOOTING, ERROR ANALYSIS, PROVIDING REPAIR SOLUTIONS, AND RANKING

(71) Applicant: Sean Earl SWIEDOM, Seattle, WA (US)

(72) Inventor: Sean Earl SWIEDOM, Seattle, WA (US)

(21) Appl. No.: 18/361,134

(22) Filed: Jul. 28, 2023

(51) Int. Cl. G16H 40/40

(52) U.S. Cl. CPC G16H 40/40

(57) ABSTRACT

The present technology provides solutions for improving efficiencies in troubleshooting and providing solutions for problems with equipment for service technicians. An example method includes receiving an input including at least one of textual data and error code type data associated with a problem for an equipment into a user interface, inputting the input into a machine learning model trained based on a data set including service repair records, identifying a particular solution of the potential solutions as the output by traversing the decision tree based on the groupings, and providing the output that facilitates the service repair operations.

### TECHNICAL FIELD

The present technology provides solutions for optimizing troubleshooting, error analysis, providing repair solutions, and ranking, and more particularly to crowdsourcing user repair data from multiple entities and leveraging a machine learning model to identify solutions.

### BACKGROUND

Equipment requires maintenance and repair over the life of the machinery. Service technicians are technicians that are trained to perform the maintenance and repair for these apparatuses. Some equipment, such as medical equipment, are often complex apparatuses that require more knowledge than the average lay person may have. Thus, the service technicians must train and learn the intricacies of the various types of equipment that they service.

Due to the need for training, service technicians for equipment, such as medical equipment, typically have a ramp up time of 6-18 months, which is a long time during which the technicians require additional oversight and teaching. In other words, it takes 6-18 months for a technician to be proficient and effective at maintenance and repairs, so that the technician can be sent to perform the necessary procedures without an additional supervisor.

### BRIEF SUMMARY

In one aspect, a method includes receiving an input including at least one of textual data and error code type data associated with a problem for an equipment into a user interface, inputting the input into a machine learning model trained based on a data set including service repair records, where the machine learning model is configured to receive the at least one of textual data and error code type data and determine an output that facilitates service repair operations for the equipment, where the service repair records are stored as anonymized, de-identified data, grouping components of the equipment into groupings, portraying potential solutions for the problem as a decision tree, identifying a particular solution of the potential solutions as the output by traversing the decision tree based on the grouping, and providing the output that facilitates the service repair operations, where the outputs include at least one of ranked solution sets, suggested actions, and suggested parts for replacement.

In another aspect, the method may also include where the machine learning model is updated based on additional service repair records stored in the shared database. Other technical features may be readily apparent to one skilled in the art from the following figures, descriptions, and claims.

In another aspect, the method may also include where each service repair record includes at least one of a type of problem, a length of time for addressing the problem, steps for troubleshooting the problem, steps for solving the problem, parts used to address the problem, and an error code associated with the problem. Other technical features may be readily apparent to one skilled in the art from the following figures, descriptions, and claims.

In another aspect, the method may also include where the machine learning model is periodically re-trained based on an amount of data in the data set. Other technical features may be readily apparent to one skilled in the art from the following figures, descriptions, and claims.

In another aspect, the method may also include where the machine learning model is configured to adjust the groupings based on particular parts or combination of parts for replacement. Other technical features may be readily apparent to one skilled in the art from the following figures, descriptions, and claims.

In one aspect, a system includes a processor. The system also includes a memory storing computer-executable instructions, which, when executed by the processor, cause the processor to perform operations includes receiving an input including at least one of textual data and error code type data associated with a problem for an equipment into a user interface, inputting the input into a machine learning model trained based on a data set including service repair records, where the machine learning model is configured to receive the at least one of textual data and error code type data and determine an output that facilitates service repair operations for the equipment, where the service repair records are stored as anonymized, de-identified data, grouping components of the equipment into groupings, portraying potential solutions for the problem as a decision tree, identifying a particular solution of the potential solutions as the output by traversing the decision tree based on the groupings, and providing the output that facilitates the service repair operations, where the outputs include at least one of ranked solution sets, suggested actions, and suggested parts for replacement.

In one aspect, a non-transitory computer-readable medium storing instructions thereon, where the instructions, when executed by one or more processors, cause the one or more processors to perform operations includes receiving an input including at least one of textual data and error code type data associated with a problem for an equipment into a user interface, inputting the input into a machine learning model trained based on a data set including service repair records, where the machine learning model is configured to receive the at least one of textual data and error code type data and determine an output that facilitates service repair operations for the equipment, where the service repair records are stored as anonymized, de-identified data, grouping components of the equipment into groupings, portraying potential solutions for the problem as a decision tree, identifying a particular solution of the potential solutions as the output by traversing the decision tree based on the groupings, and providing the output that facilitates the service repair operations, where the outputs include at least one of ranked solution sets, suggested actions, and suggested parts for replacement.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an example system for troubleshooting, error analysis, providing repair solutions, and ranking in accordance with one embodiment.

FIG. 2 illustrates an example method for troubleshooting, error analysis, providing repair solutions, and ranking in accordance with one embodiment.

FIG. 3 illustrates an example lifecycle of a machine learning model in accordance with one embodiment.

FIG. 4 shows an example of a system for implementing certain aspects of the present technology.

### DETAILED DESCRIPTION

Equipment requires maintenance and repair over the life of the machinery. Service technicians are technicians that are trained to perform the maintenance and repair for these apparatuses. Some equipment, such as medical equipment, are often complex apparatuses that require more knowledge than the average lay person may have. Thus, the service technicians must train and learn the intricacies of the various types of equipment that they service.

Due to the need for training, service technicians for equipment, such as medical equipment, typically have a ramp up time of 6-18 months, which is a long time during which the technicians require additional oversight and teaching. In other words, it takes 6-18 months for a technician to be proficient and effective at maintenance and repairs, so that the technician can be sent to perform the necessary procedures without an additional supervisor.

Additionally, the availability of service technicians is decreasing due to retirees and a slowing number of people moving into this particular sector in the industry to perform these duties. This causes the current service technicians to work long hours. For example, the average service technician can often work upwards of 50 hours per week, which stresses both the technicians and the organizations. These hours are often filled with inefficient troubleshooting and repair. Furthermore, training of technicians does not alleviate enough of the knowledge deficit based on the extended training period as discussed above.

Moreover, solutions to each problem of the machinery is often learned by experienced technicians over time, rather than having a regular and consistent solution. A major contributor to the long duration for training is the relative scarcity of frequent problems and the large diversity or variety of problems that can occur for each different piece of equipment. For example, hospital systems may encounter particular repair problems a couple times per year. In other words, due to the lack of consistency and infrequency of types of problems, repairs, and/or solutions, it is difficult for a service technician to learn the required skills to be proficient at performing the repairs and maintenance without an additional experienced supervisor. Thus, a particular hospital system would require years and years of repairs to develop a large data set of solutions and repairs.

Current technology often includes lengthy and complex instructions to train technicians. However, these instructions are often expensive and difficult to maintain due to the complexity, amount of information or detail, and inability to rank solutions and more frequent problems.

The present technology provides solutions for a system and method configured to accelerate the troubleshooting and repair process of equipment. More specifically, the present technology minimizes time required for troubleshooting and repair, while also minimizing the amount of time required for a service technician to be trained. In other words, the benefits of the present technology are twofold: 1) acceleration of training time for technicians; and 2) streamlining troubleshooting and repair of equipment.

First, the present technology can provide additional context and facilitate selection of frequent problems, so that the steps and parts required for repairing the frequent problems are provided first to training technicians. Second, the present technology provides an efficient system and method for troubleshooting problems and identifying necessary repairs and replacement parts.

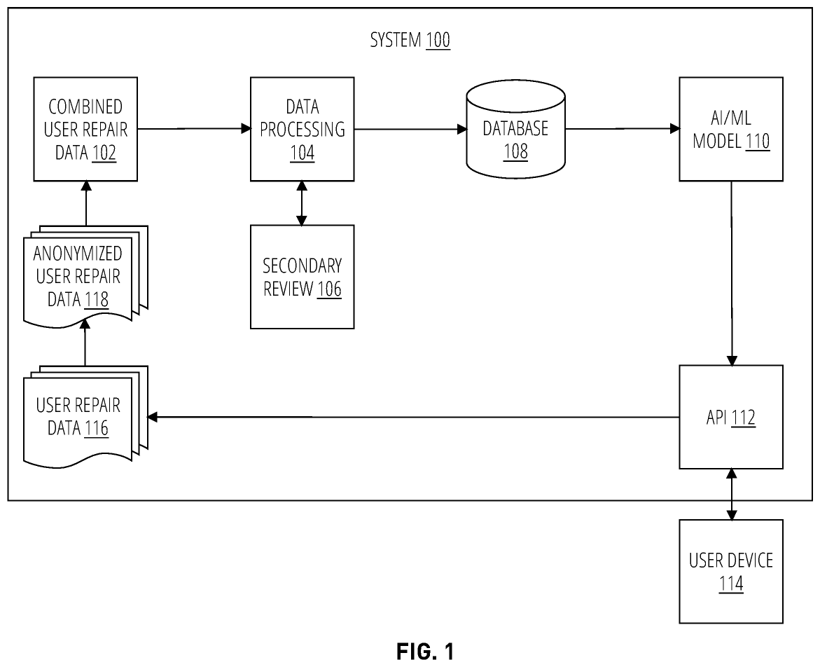

FIG. 1 illustrates an environment having a system 100 that includes combined user repair data 102, data processing 104, secondary review 106, a database 108, an AI/ML model 110, an application programming interface (API) 112, a user device 114, user repair data 116, and anonymized user repair data 118.

Combined user repair data 102 is an aggregation of user repair data for equipment, such as medical equipment. More specifically combined user repair data 102 includes service repair records, which can contain information related to repairs performed by technicians to address relevant problems, such as breakdowns, broken parts, etc. Combined user repair data 102 can be aggregated from a plurality of different users across different entities. For example, combined user repair data 102 can be collected from technicians across different hospital systems. Thus, combined user repair data 102 can have more data than a singular system may have. Accordingly, combined user repair data 102 can be crowdsourced data received from a plurality of users in some embodiments.

User repair data can include symptoms of a problem, repairs, procedures, steps, replacement parts, error codes, and other relevant information for correcting the problem for that particular equipment. More specifically, each service repair record includes at least one of a type of problem, a length of time for addressing the problem, steps for troubleshooting the problem, steps for solving the problem, parts used to address the problem, and an error code associated with the problem. Additionally, these different types of relevant information can be used to facilitate problems explained by user inputted symptoms. In other words, a system of the present technology is configured to receive symptoms of a problem inputted by a user and compare the symptoms with the information in combined user repair data 102 to identify a solution for the problem.

For example, a technician may input that a Magnetic Resonance Imaging (MRI) machine is producing a clunking noise during a process that should not produce a clunking noise. A technician can provide an input (e.g., a textual input) including at least one type of relevant information identifying the clunking noise for the MRI machine during the process. The system can then receive the input and provide an output based on the textual input.

A data processing 104 module or system can process combined user repair data 102 to be later stored in database 108. Data processing 104 can include data ingestion, sanitization, extraction, classification, categorization, association, de-identifying, anonymizing, and other similar processing procedures. For example, data processing 104 can include extracting parts and steps performed identified in the service repair records, associating the parts and steps of the repair with an error code for the problem, classifying a repair with a difficulty level, and categorizing the problem and/or repair based on any of the above.

In some embodiments, data processing 104 can be automated. More specifically, a system can be configured to receive data in a particular format, so that the system can automatically extract data. For example, a system can be configured to receive textual data, so that data can be extracted based on an order of inputs, length of text, punctuation or other specific characters, etc. In some embodiments, data processing 104 can be conducted by a textual AI/ML model that is configured to interpret text, extract the information, and store the information in database 108 accordingly. In some embodiments, data processing 104 can also be configured to filter and validate good data (e.g., data that is accurate) and remove bad data (e.g., data that is inaccurate). For example, data processing 104 may determine that data from a service repair record includes various typographical errors. In some embodiments, the data may include photographic or image data and data processing 104 can include an image recognition AI/ML model configured to extract data and information from the image and store and/or output the data and information to database 108.

Secondary review 106 can be performed to further improve data processing 104. In some embodiments, secondary review 106 is performed to facilitate data ingestion and processing when an automated system is unable to process the data and/or flags the data for secondary review. For example, the system may identify that some data may be bad data, which should not be included into database 108. Thus, the system can request secondary review 106 for the data (e.g., by a human, and/or an AI/ML model). The data can then either be removed or re-added to the data set for inclusion in database 108.

Database 108 stores processed data (e.g., user repair data after data processing 104). In some embodiments, database 108 can be configured to identify associations between problems, solutions, and other aspects of the data. Database 108 can be configured as a relational database, a distributed database, a consolidated database, a shared database, and other forms of data stores. Furthermore, database 108 can be configured to store the processed data as de-identified, anonymized data for any privacy concerns.

AI/ML model 110 is trained based on data received from database 108. More specifically, AI/ML model 110 is configured to receive symptoms or other relevant information (e.g., as textual input) of a problem and identify and/or provide an output that is used to solve the problem. In other words, AI/ML model 110 is configured to receive textual data or input including a symptom or error code for a problem and determine an output that facilitates service repair operations for the equipment. In some embodiments, AI/ML model 110 can also receive feedback data identifying whether the provided output facilitated repair for the problem. Additionally, in some embodiments, AI/ML model 110 can be trained by encouraging input and output pairs based on the feedback data.

More specifically, AI/ML model 110 can identify potential solutions (e.g., repair steps, replacement parts, etc.) as a decision tree. The AI/ML model 110 can traverse the decision tree based on the input and/or textual data. For example, AI/ML model 110 can utilize observation of a particular symptom or identification of a particular grouping to select one branch of the decision tree over another branch until reaching a solution. In some embodiments, each additional decision increases a confidence level for a particular solution, such that the particular solution is selected as the output when the confidence level is above a threshold level.

In some embodiments, AI/ML model 110 can group components of equipment into groupings. The groupings can be used as classifications and/or categories. For example, components of a machine that are in close proximity with one another may be grouped together into one grouping. Accordingly, the groupings can be used to more holistically identify a component rather than specifying particular nuts and bolts for replacement parts. In some embodiments, AI/ML model 110 can be configured to adjust the groupings based on frequency of each component in particular repairs.

Additionally, AI/ML model 110 can determine a frequency or total number of queries that identify a particular component or grouping of components. The AI/ML model 110 can utilize frequency or total number to identify components that are more prone to failure or otherwise require maintenance and/or repair. In some embodiments, AI/ML model 110 can provide the frequency along with other information in the output to the user. In some embodiments, the AI/ML model 110 can be leveraged as a training tool to provide information for maintaining and repairing various systems for a respective facility. For example, AI/ML model 110 can provide repair steps and required replacement parts for more frequent problems, so that a training technician can learn the solutions to more frequent problems and be more proficient and effective at the type of repairs and maintenance that are most commonly performed. In other words, AI/ML model 110 can expedite training of technicians by providing training materials for the more common issues. The AI/ML model 110 can also be configured to predict parts consumption, repair time, and a difficulty associated with the output.

Furthermore, AI/ML model 110 can be periodically retrained or continuously learning. For example, AI/ML model 110 can be updated with additional user repair data 116 that is received over time periodically or continuously.

API 112 provides an interface for a user device 114 to provide a query or input to AI/ML model 110. For example, a technician using user device 114 can send a query or input identifying symptoms of a problem for a machine through API 112 and receive an output from AI/ML model 110.

User device 114 is a client device associated with a user. A client device can be a terminal, a computer, a server, a mobile device, a tablet, a cellphone, or any other suitable device capable of accessing API 112.

User repair data 116 is data received from user device 114. More specifically, user repair data 116 includes feedback regarding accuracy and/or helpfulness for the output generated by AI/ML model 110 and user inputted data for service repair records as discussed above. For example, a technician may indicate that the steps and parts identified in the output from AI/ML model 110 was accurate. As another example, a technician may provide information in service repair records as user repair data 116 to provide additional data for AI/ML model 110.

Anonymized user repair data 118 is user repair data 116 after being anonymized. In other words, user repair data 116 is sanitized of entity identifying information to generate anonymized user repair data 118, which can then be aggregated into combined user repair data 102.

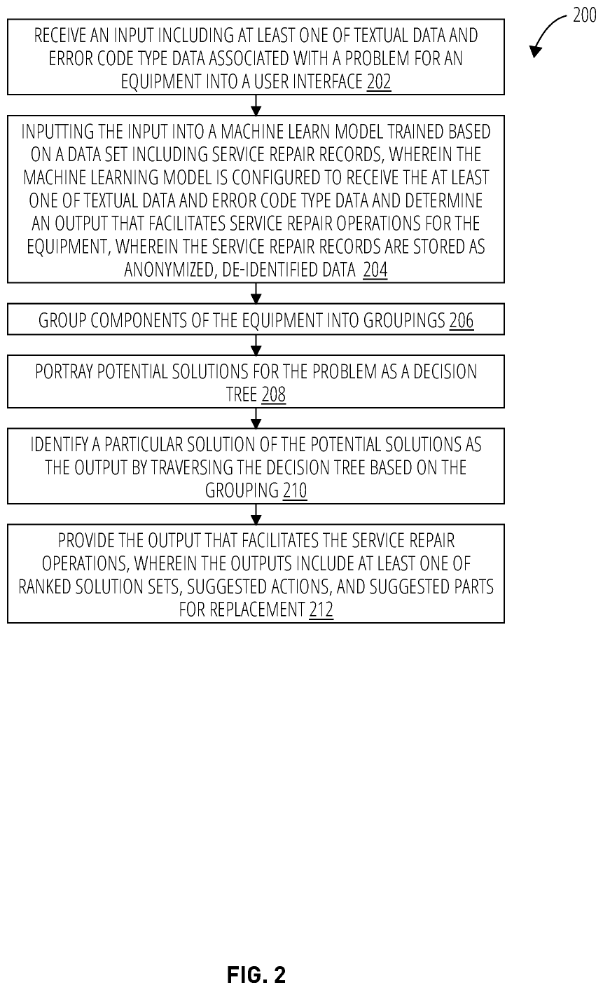

FIG. 2 illustrates an example method 200 for troubleshooting, error analysis, providing repair solutions, and ranking. Although the example method 200 depicts a particular sequence of operations, the sequence may be altered without departing from the scope of the present disclosure. For example, some of the operations depicted may be performed in parallel or in a different sequence that does not materially affect the function of the method 200. In other examples, different components of an example device or system that implements the method 200 may perform functions at substantially the same time or in a specific sequence.

In step 202, method 200 receives an input including at least one of textual data and error code type data associated with a problem for an equipment into a user interface. For example, system 100 (e.g., by API 112) can receive an input including at least one of textual data and error code type data associated with a problem for an equipment from a user interface.

In step 204, method 200 inputting the input into a machine learning model trained based on a data set including service repair records, wherein the machine learning model is configured to receive the at least one of textual data and error code type data and determine an output that facilitates service repair operations for the equipment, wherein the service repair records are stored as anonymized, de-identified data. For example, system 100 (e.g., via AI/ML model 110 and/or API 112) inputs the input into a machine learning model.

In step 206, method 200 groups components of the equipment into groupings. For example, system 100 (e.g., via AI/ML model 110) can group components of the equipment into groupings. The groupings can be used to facilitate traversal of a decision tree.

In step 208, method 200 portrays potential solutions for the problem as a decision tree. For example, system 100 can portray potential solutions for the problem as a decision tree.

In step 210, method 200 identifies a particular solution of the potential solutions as the output by traversing the decision tree based on the grouping. For example, system 100 (e.g., by AI/ML model 110) can identify a particular solution of the potential solutions as the output by traversing the decision tree based on the grouping.

In step 212, method 200 provides the output that facilitates the service repair operations, wherein the outputs include at least one of ranked solution sets, suggested actions, and suggested parts for replacement. For example, system 100 (e.g., by API 112) provides the output that facilitates the service repair operations.

Additionally, the method 200 can further receive feedback information regarding accuracy of the output from a user device. For example, system 100 (e.g., via API 112) can receive the feedback information. The feedback information can then be used to update AI/ML model 110.

Additionally or alternatively, the method 200 can further receive additional user repair data 116 identifying steps and parts and other associated information for solving the problem to further update AI/ML model 110 as further discussed below.

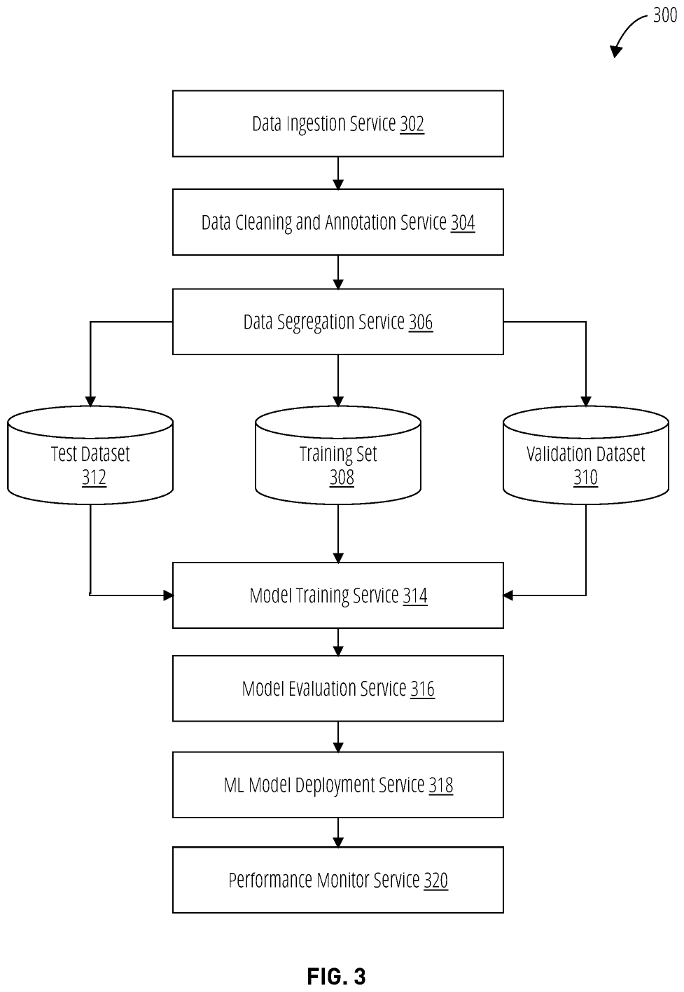

FIG. 3 illustrates an example lifecycle 300 of a ML model in accordance with some examples. The first stage of the lifecycle 300 of a ML model is a data ingestion service 302 to generate datasets described below. ML models require a significant amount of data for the various processes described in FIG. 3 and the data persisted without undertaking any transformation to have an immutable record of the original dataset. The data can be provided from third party sources such as publicly available dedicated datasets. The data ingestion service 302 provides a service that allows for efficient querying and end-to-end data lineage and traceability based on a dedicated pipeline for each dataset, data partitioning to take advantage of the multiple servers or cores, and spreading the data across multiple pipelines to reduce the overall time to reduce data retrieval functions.

In some cases, the data may be retrieved offline that decouples the producer of the data from the consumer of the data (e.g., an ML model training pipeline). For offline data production, when source data is available from the producer, the producer publishes a message and the data ingestion service 302 retrieves the data. In some examples, the data ingestion service 302 may be online and the data is streamed from the producer in real-time for storage in the data ingestion service 302.

After data ingestion service 302, a data preprocessing service preprocesses the data to prepare the data for use in the lifecycle 300 and includes at least data cleaning, data transformation, and data selection operations. The data cleaning and annotation service 304 removes irrelevant data (data cleaning) and general preprocessing to transform the data into a usable form. The data cleaning and annotation service 304 includes labelling of features relevant to the ML model. In some examples, the data cleaning and annotation service 304 may be a semi-supervised process performed by a ML to clean and annotate data that is complemented with manual operations such as labeling of error scenarios, identification of untrained features, etc.

After the data cleaning and annotation service 304, data segregation service 306 to separate data into at least a training set 308, a validation dataset 310, and a test dataset 312. Each of the training set 308, a validation dataset 310, and a test dataset 312 are distinct and do not include any common data to ensure that evaluation of the ML model is isolated from the training of the ML model.

The training set 308 is provided to a model training service 314 that uses a supervisor to perform the training, or the initial fitting of parameters (e.g., weights of connections between neurons in artificial neural networks) of the ML model. The model training service 314 trains the ML model based a gradient descent or stochastic gradient descent to fit the ML model based on an input vector (or scalar) and a corresponding output vector (or scalar).

After training, the ML model is evaluated at a model evaluation service 316 using data from the validation dataset 310 and different evaluators to tune the hyperparameters of the ML model. The predictive performance of the ML model is evaluated based on predictions on the validation dataset 310 and iteratively tunes the hyperparameters based on the different evaluators until a best fit for the ML model is identified. After the best fit is identified, the test dataset 312, or holdout data set, is used as a final check to perform an unbiased measurement on the performance of the final ML model by the model evaluation service 316. In some cases, the final dataset that is used for the final unbiased measurement can be referred to as the validation dataset and the dataset used for hyperparameter tuning can be referred to as the test dataset.

After the ML model has been evaluated by the model evaluation service 316, an ML model deployment service 318 can deploy the ML model into an application or a suitable device. The deployment can be into a further test environment such as a simulation environment, or into another controlled environment to further test the ML model.

After deployment by the ML model deployment service 318, a performance monitor service 320 monitors for performance of the ML model. In some cases, the performance monitor service 320 can also record additional transaction data that can be ingested via the data ingestion service 302 to provide further data, additional scenarios, and further enhance the training of ML models.

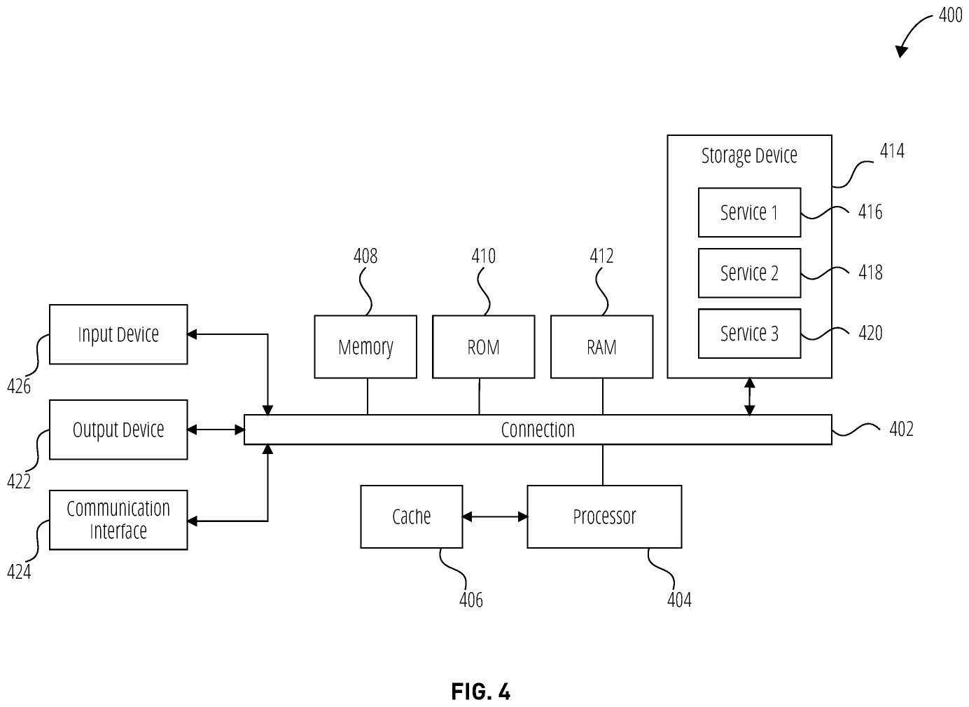

FIG. 4 shows an example of computing system 400, which can be for example any computing device making up system 100 or any component thereof in which the components of the system are in communication with each other using connection 402. Connection 402 can be a physical connection via a bus, or a direct connection into processor 404, such as in a chipset architecture. Connection 402 can also be a virtual connection, networked connection, or logical connection.

In some embodiments, computing system 400 is a distributed system in which the functions described in this disclosure can be distributed within a datacenter, multiple data centers, a peer network, etc. In some embodiments, one or more of the described system components represents many such components each performing some or all of the function for which the component is described. In some embodiments, the components can be physical or virtual devices.

Example computing system 400 includes at least one processing unit (CPU or processor) 404 and connection 402 that couples various system components including system memory 408, such as read-only memory (ROM) 410 and random access memory (RAM) 412 to processor 404. Computing system 400 can include a cache of high-speed memory 406 connected directly with, in close proximity to, or integrated as part of processor 404.

Processor 404 can include any general purpose processor and a hardware service or software service, such as services 416, 418, and 420 stored in storage device 414, configured to control processor 404 as well as a special-purpose processor where software instructions are incorporated into the actual processor design. Processor 404 may essentially be a completely self-contained computing system, containing multiple cores or processors, a bus, memory controller, cache, etc. A multi-core processor may be symmetric or asymmetric.

To enable user interaction, computing system 400 includes an input device 426, which can represent any number of input mechanisms, such as a microphone for speech, a touch-sensitive screen for gesture or graphical input, keyboard, mouse, motion input, speech, etc. Computing system 400 can also include output device 422, which can be one or more of a number of output mechanisms known to those of skill in the art. In some instances, multimodal systems can enable a user to provide multiple types of input/output to communicate with computing system 400. Computing system 400 can include communication interface 424, which can generally govern and manage the user input and system output. There is no restriction on operating on any particular hardware arrangement, and therefore the basic features here may easily be substituted for improved hardware or firmware arrangements as they are developed. An input device 426 can include a camera sensor configured to capture photos. For example, a camera sensor of a mobile device can capture a photo. The photo can be processed (e.g., with the assistance of AI/ML model 110) to extract and determine text and/or error codes in the photo.

Storage device 414 can be a non-volatile memory device and can be a hard disk or other types of computer readable media which can store data that are accessible by a computer, such as magnetic cassettes, flash memory cards, solid state memory devices, digital versatile disks, cartridges, random access memories (RAMs), read-only memory (ROM), and/or some combination of these devices.

The storage device 414 can include software services, servers, services, etc., that when the code that defines such software is executed by the processor 404, it causes the system to perform a function. In some embodiments, a hardware service that performs a particular function can include the software component stored in a computer-readable medium in connection with the necessary hardware components, such as processor 404, connection 402, output device 422, etc., to carry out the function.

For clarity of explanation, in some instances, the present technology may be presented as including individual functional blocks including functional blocks comprising devices, device components, steps or routines in a method embodied in software, or combinations of hardware and software.

Any of the steps, operations, functions, or processes described herein may be performed or implemented by a combination of hardware and software services or services, alone or in combination with other devices. In some embodiments, a service can be software that resides in memory of a client device and/or one or more servers of a content management system and perform one or more functions when a processor executes the software associated with the service. In some embodiments, a service is a program or a collection of programs that carry out a specific function. In some embodiments, a service can be considered a server. The memory can be a non-transitory computer-readable medium.

In some embodiments, the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer-readable media. Such instructions can comprise, for example, instructions and data which cause or otherwise configure a general purpose computer, special purpose computer, or special purpose processing device to perform a certain function or group of functions. Portions of computer resources used can be accessible over a network. The executable computer instructions may be, for example, binaries, intermediate format instructions such as assembly language, firmware, or source code. Examples of computer-readable media that may be used to store instructions, information used, and/or information created during methods according to described examples include magnetic or optical disks, solid-state memory devices, flash memory, USB devices provided with non-volatile memory, networked storage devices, and so on.

Devices implementing methods according to these disclosures can comprise hardware, firmware and/or software, and can take any of a variety of form factors. Typical examples of such form factors include servers, laptops, smartphones, small form factor personal computers, personal digital assistants, and so on. The functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

What is claimed is:

1. A method comprising:
receiving an input including at least one of textual data and error code type data associated with a problem for an equipment into a user interface;
inputting the input into a machine learning model trained based on a data set including service repair records, wherein the machine learning model is configured to receive the at least one of textual data and error code type data and determine an output that facilitates service repair operations for the equipment, wherein the service repair records are stored as anonymized, de-identified data;
grouping components of the equipment into groupings;
portraying potential solutions for the problem as a decision tree;
identifying a particular solution of the potential solutions as the output by traversing the decision tree based on the groupings; and
providing the output that facilitates the service repair operations, wherein the outputs include at least one of ranked solution sets, suggested actions, and suggested parts for replacement.

2. The method of claim 1, wherein the service repair records are crowdsourced data from different users and stored in a shared database.

3. The method of claim 2, wherein the machine learning model is updated based on additional service repair records stored in the shared database.

4. The method of claim 1, wherein each service repair record includes at least one of a type of problem, a length of time for addressing the problem, steps for troubleshooting the problem, steps for solving the problem, parts used to address the problem, and an error code associated with the problem.

5. The method of claim 1, wherein the machine learning model is periodically re-trained based on an amount of data in the data set.

6. The method of claim 1, wherein the machine learning model is configured to adjust the groupings based on particular parts or combination of parts for replacement.

7. The method of claim 1, wherein the machine learning model is further configured to predict parts consumption, repair time, and a difficulty associated with the output.

8. A system comprising:
a processor; and
a memory storing computer-executable instructions, which, when executed by the processor, cause the processor to perform operations comprising:
receiving an input including at least one of textual data and error code type data associated with a problem for an equipment into a user interface;
inputting the input into a machine learning model trained based on a data set including service repair records, wherein the machine learning model is configured to receive the at least one of textual data and error code type data and determine an output that facilitates service repair operations for the equipment, wherein the service repair records are stored as anonymized, de-identified data;
grouping components of the equipment into groupings;
portraying potential solutions for the problem as a decision tree;
identifying a particular solution of the potential solutions as the output by traversing the decision tree based on the groupings; and
providing the output that facilitates the service repair operations, wherein the outputs include at least one of ranked solution sets, suggested actions, and suggested parts for replacement.

9. The system of claim 8, wherein the service repair records are crowdsourced data from different users and stored in a shared database.

10. The system of claim 9, wherein the machine learning model is updated based on additional service repair records stored in the shared database.

11. The system of claim 8, wherein each service repair record includes at least one of a type of problem, a length of time for addressing the problem, steps for troubleshooting the problem, steps for solving the problem, parts used to address the problem, and an error code associated with the problem.

12. The system of claim 8, wherein the machine learning model is periodically re-trained based on an amount of data in the data set.

13. The system of claim 8, wherein the machine learning model is configured to adjust the groupings based on particular parts or combination of parts for replacement.

14. The system of claim 8, wherein the machine learning model is further configured to predict parts consumption, repair time, and a difficulty associated with the output.

15. A non-transitory computer-readable medium storing instructions thereon, wherein the instructions, when executed by one or more processors, cause the one or more processors to perform operations comprising:
receiving an input including at least one of textual data and error code type data associated with a problem for an equipment into a user interface;
inputting the input into a machine learning model trained based on a data set including service repair records, wherein the machine learning model is configured to receive the at least one of textual data and error code type data and determine an output that facilitates service repair operations for the equipment, wherein the service repair records are stored as anonymized, de-identified data;
grouping components of the equipment into groupings;
portraying potential solutions for the problem as a decision tree;
identifying a particular solution of the potential solutions as the output by traversing the decision tree based on the groupings; and
providing the output that facilitates the service repair operations, wherein the outputs include at least one of ranked solution sets, suggested actions, and suggested parts for replacement.

16. The non-transitory computer-readable medium of claim 15, wherein the service repair records are crowdsourced data from different users and stored in a shared database.

17. The non-transitory computer-readable medium of claim 16, wherein the machine learning model is updated based on additional service repair records stored in the shared database.

18. The non-transitory computer-readable medium of claim 15, wherein each service repair record includes at least one of a type of problem, a length of time for addressing the problem, steps for troubleshooting the problem, steps for solving the problem, parts used to address the problem, and an error code associated with the problem.

19. The non-transitory computer-readable medium of claim 15, wherein the machine learning model is periodically re-trained based on an amount of data in the data set.

20. The non-transitory computer-readable medium of claim 15, wherein the machine learning model is configured to adjust the groupings based on particular parts or combination of parts for replacement.
